(19) United States
(12) Patent Application Publication
(10) Pub. No.: US 2007/0233160 A1
(43) Pub. Date: Oct. 4, 2007

(54) HEAD FOR ELECTRICALLY OPERATED TYPE EPILATING DEVICE

(76) Inventor: Tae-Jun Oh, Gyeonggi-do (KR)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/693,979

(22) Filed: Mar. 30, 2007

(30) Foreign Application Priority Data
Apr. 3, 2006 (KR) UM20-2006-0008724

(51) Int. Cl. A61B 17/50

(52) U.S. Cl. 606/133

(57) ABSTRACT

The present invention relates to an electrically operated type of epilating device, and more particularly to an electrically operated type epilating device that has improved pinching plates and is capable of effectively pinching user's hair by placing an intermediate member having an intermediate pinching plate between the pinching plates. The epilating device comprises a main body housing, a motor, a motor gear, and a driving gear. A head includes a pair of rotating plates being engaged and rotated with the driving gear; a plurality of sets of epilating units being arranged between the rotating plates, each epilating unit including first pinching plate having three wing portions formed to be protruded outwardly and a through-hole at center portion thereof, a second pinching plate having the same construction that of the first pinching plate, and an intermediate member being arranged between the first pinching plate and the second pinching plates; first pusher members pressing inwardly the first pinching plates; second pusher members being arranged to be overlapped with lower portions of the first push members and pressing the second pinching plates in opposing direction to the first pinching plates; first and second rollers for pressing in opposing directions; a pair of fixing plates for rotatably installing the rollers; and a fixing lever for preventing the fixing plate from rotating.

### CROSS-REFERENCE TO RELATED APPLICATIONS

This application claims priority to Korean Patent Application No. UM 20-2006-0008724 entitled “HEAD FOR ELECTRICALLY OPERATED TYPE EPILATING DEVICE” filed on Apr. 3, 2006.

### FIELD OF THE INVENTION

The present invention relates to an electrically operated type epilating device, and more particularly to an electrically operated type epilating device, which has improved pinching plates and is capable of effectively pinching user's hair by arranging an intermediate member having an intermediate pinching plate between the pinching plates.

### BACKGROUND ART

In a conventional electrically operated type epilating device, a head includes plural sets of two pinching circular plates installed to a reversed eyebrow-shaped flexible shaft, so that one set of two pinching plates is placed in a reversed “V” form. Here, user's hair is pinched and pulled out by a plural sets of pinching plates in a head, in the course of advancing to a point where each set of pinching plates are contacted with each other. That is, the epilating device has generally pairs of steel plates, and a user's hair is pinched and pulled out by the steel plates as the steel plates are tightly contacted with and departed from each other, or the user's hair is cut and pulled out when a mesh, into which hair is inserted, is tightly contacted and moved with the steel plates. However, since the strength of hair is very weak, user's hair may be not pinched by the pinching plates or the pinched hair tends to be released from the pinching plates due to the contact resistance of vibrations and skin.

Further, there is known another type of epilating device, which is capable of pinching a user's hair by pressing the pinching plates in opposing direction with a pusher member to deform them in a horizontal line form or a crosswise form. However, in case of the horizontal line form of pinching plates, the conventional epilating device has disadvantage in that many parts are required to thereby complicate the structure and thus the manufacture cost becomes expensive, since a plurality of pinching plates are required along a rotation direction thereof. Also, in case of the crosswise form of pinching plates, it has disadvantages in that the pinching force to user's hair is weakened, since user's hair is pinched by only adjacent two plates, and the number of pulled out hair is relatively less upon one revolution of the head, since wing portions of the head are arranged at intervals of 90°.

### SUMMARY OF THE INVENTION

An object of the present invention is to solve problems of conventional epilating devices and to provide an electrically operated type epilating device having a stronger pinching force to user's hair and being capable of removing much user's hair upon one revolution of a head thereof.

The object and advantages of the present invention will be described in detail referring to following embodiments and will be realized by means described in claims and by combinations thereof.

To achieve this object, an electrically operated type epilating device in accordance with the invention is characterized in that the electrically operated type epilating device comprises a main body housing for containing a motor; a head fixing case being mounted to an upper portion of the main body housing for fixing a head, and being opened inwardly to expose the head at top thereof; a motor gear being rotated by the motor; an intermediate gear being engaged with the motor gear; and a driving gear being engaged and rotated with the intermediate gear; wherein the head includes: a pair of rotating plates having teeth engaged and rotated with the driving gear at outer periphery thereof and a hollow shaft at center portion thereof into which a center rod is inserted and passing through it, and through-holes drilled at intervals of 60° around a center of the hollow shaft; a plurality of epilating units being arranged between the rotating plates, each epilating unit including a first pinching plate having a first through-hole a at center portion thereof into which the center rod is inserted, three wing portions formed to be protruded outwardly at the first pinching plate at intervals of 120° around the through-hole, and a second through-hole drilled at a center of the wing portions, the second pinching plate having the same shape as the first pinching plate, and an intermediate member being arranged between the first pinching plate and the second pinching plate; a first pusher members being in turn fitted into the rotating plates and the epilating units, and pressing inwardly the first pinching plates; a second pusher members being arranged to be overlapped with lower portions of the first push members, and being fitted in turn into the rotating plates and the epilating unit, pressing the second pinching plates in opposing direction to the first pinching plates; a roller for pressing the first push members and the second pusher members; a pair of fixing plates including roller installation holes for installing the rollers rotatably, and being fixed at ends of the center rod; and a fixing lever for pressing the fixing plates from outside in order to prevent the fixing plate from rotating, and being inserted and fixed into the head fixing case.

### BRIEF DESCRIPTION OF THE DRAWINGS

The novel features believed characteristic of the invention are set forth in the appended claims. The invention itself, however, as well as a preferred mode of use, further objectives and advantages thereof, will best be understood by reference to the following detailed description of an illustrative embodiment when read in conjunction with the accompanying drawings, wherein:

FIG. 1 is an outer perspective view of an electrically operated type epilating device according to the present invention;

FIG. 2 is an inner perspective view of an electrically operated type epilating device according to the present invention;

FIG. 3 is a cross-sectional view of an electrically operated type epilating device according to the present invention;

FIG. 4 is an exploded perspective view of an electrically operated type epilating device according to the present invention;

FIG. 5 is an exploded perspective view of epilating units in an electrically operated type epilating device according to the present invention;

FIG. 6 is an exploded perspective view showing epilating units and a pusher member, which are not engaged with each other, in an electrically operated type epilating device according to the present invention;

FIG. 7 is an exploded perspective view showing epilating units and a pusher member, which are engaged with each other, in an electrically operated type epilating device according to the present invention; and

FIGS. 8a and 8b are cross-sectional views for illustrating operating states of an electrically operated type epilating device according to the present invention, respectively.

### DETAILED DESCRIPTION

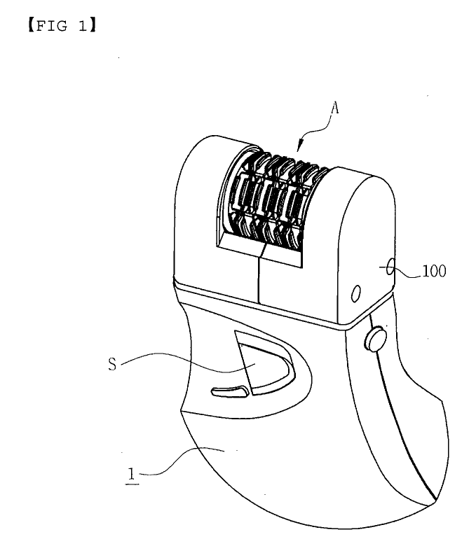

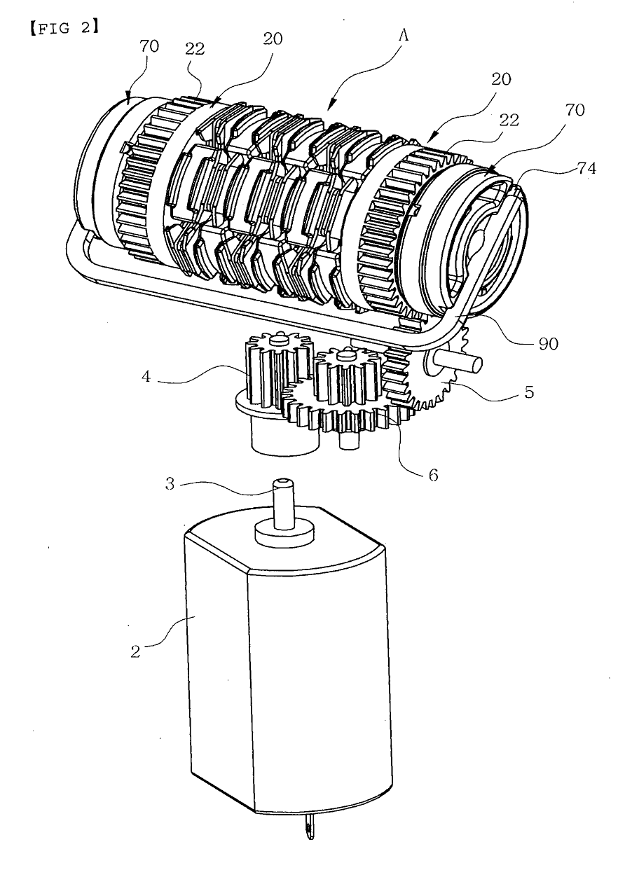

As shown in FIGS. 1 to 4, an electrically operated type epilating device according to the present invention comprises a main body housing 1, a motor 2, a motor gear 4, a driving gear 5, an intermediate gear 6, and a head A.

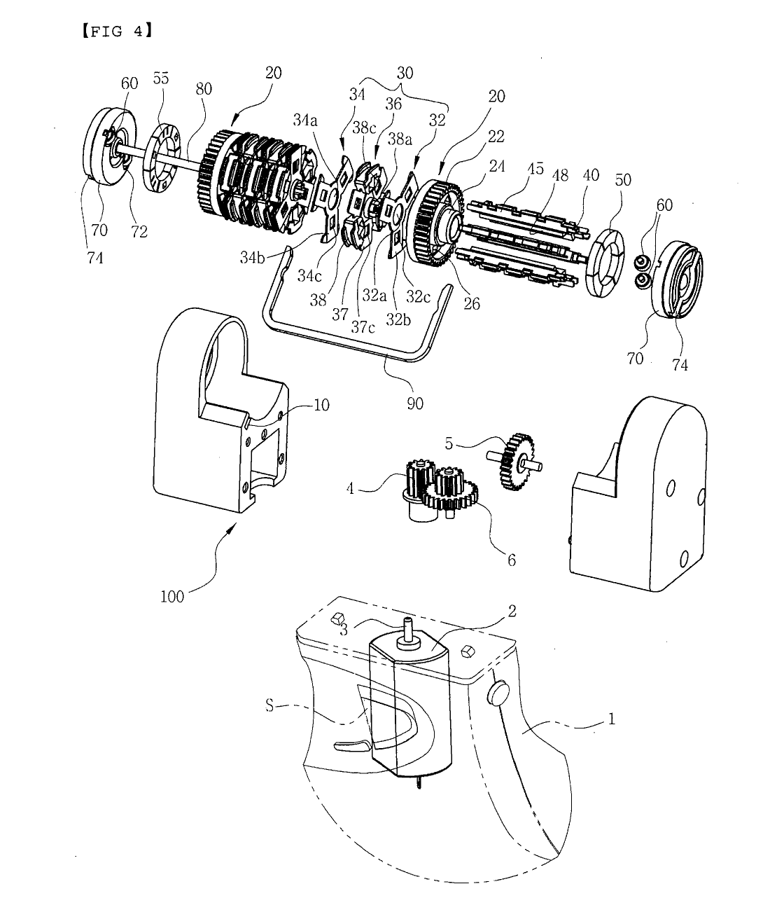

The main body housing 1 is a box in which a variety of parts such as the motor 2, battery and like, as described below, are contained. A head fixing case 100 for fixing the head A is mounted to an upper portion of the main body housing 1. The head fixing case 100 is opened inwardly to expose the head A at top thereof. The head fixing case 100 can be divided into right and left sides to be engaged with each other and be disengaged from each other as shown in FIG. 1 or 4.

As described above, the main body housing 1 is provided with the motor 2, the motor gear 4 is engaged with an outer periphery of a motor shaft 3 of the motor 2, so that the motor gear 4 is rotated along with the motor shaft 3.

The motor gear 4 is engaged with the intermediate gear 6, and then the intermediate gear 6 is engaged with the driving gear 5. Therefore, if the motor 4 is rotated by an operation of a switch S, a rotating force produced by the motor 4 is transferred to the driving gear 5 through the intermediate gear 6, so that a pair of rotating plates 20 engaged with the driving gear 5 are rotated to cause the head A to rotate. Such gear structure is well known, and thus the detailed description thereof is omitted herein for clarity of description.

The head A of the of epilating device according to the present invention comprises the rotating plates 20, epilating units 30, first pusher members 40, second pusher members 45, and fixing plates 70.

The rotating plates 20 are of a circular disk shape and are disposed in the right and left sides, respectively. The rotating plates 20 are engaged and rotated with the driving gear 5 as above described to operate the head A, and thus an outer periphery of the rotating plates 20 is provided with teeth 22 for engaging with the driving gear 5. The rotating plates 20 are provided with a hollow shaft 24 at a center portion thereof, respectively. A center rod 80 is inserted into the hollow shaft 24 and passing through it. Six through-holes 26 are drilled at intervals of 60° around a center of the hollow shaft 24, so that the first pusher members 40, the second pusher members 45, and fixing bars 48 can be inserted into the through-holes 26.

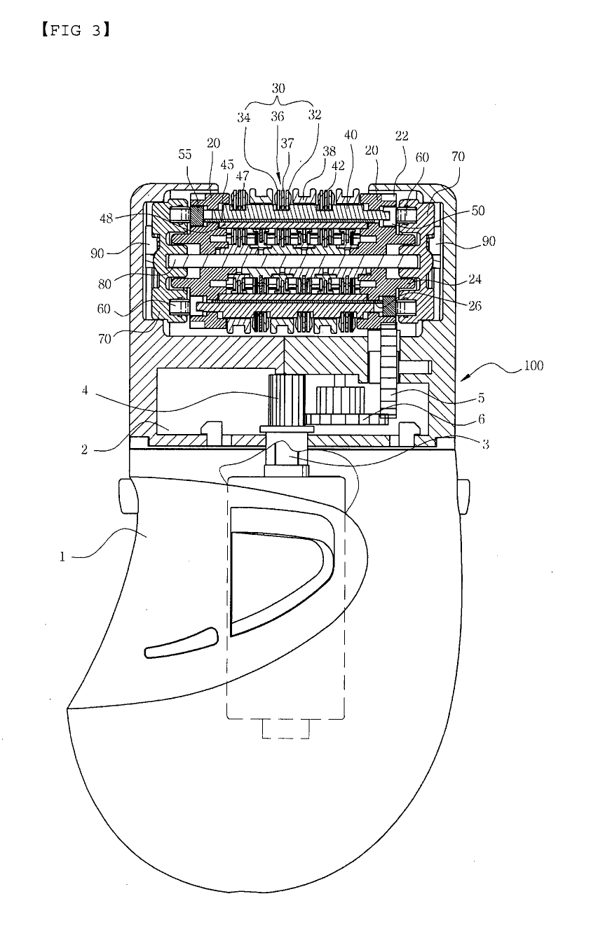

The epilating units 30 are arranged between the rotating plates 20. The epilating units 30 are rotated along with the rotating plates 20, and at the same time, are pressed by the first pusher members 40 and the second pusher members 45 to pull out user's hair. As shown in FIGS. 3 and 4, the epilating units 30 comprises first pinching plates 32, second pinching plates 34, and intermediate members 36.

The first pinching plates 32 are plate member made from metal material and has a through-hole 32a at center portion thereof, into which the center rod 80 is inserted. Three wing portions 32b are formed to be protruded outwardly at the first pinching plate 32 at intervals of 120° around the through-hole 32a. Another through-hole 32c is drilled at a center of each wing portion 32b, so that the first pusher members 40, the second pusher members 45, and fixing bars 48 are also inserted into the through-hole 32c to pass through it. Material and shape of the second pinching plate 34 is the same as those of the first pinching plate 32, and also the orientation and shape of the wing portions of the second pinching plates 34 are the same as those of the first pinching plate 32.

The intermediate members 36 are inserted into the first pinching plate 32 and the second pinching plates 34. The intermediate members 36 includes intermediate pinching plates 37 having the same shape and orientation as the first pinching plate 32 and the second pinching plates 34, and a plastic body 38 surrounding a central portion of the intermediate pinching plate 37 from outside. The intermediate member 36 is formed by an insert injection molding process. That is, if the intermediate pinching plate 37 is inserted into a mold, then melted plastic material is filled into the mold to form the intermediate member 36. Therefore, the central portion of the intermediate pinching plate 37 is surrounded by the plastic body 38, but the wing portions are exposed. Further, the plastic body 38 is provided with protruding portions between the wing portions of the intermediate pinching plate 37. Through-holes 38a are formed at a central portion of the plastic body 38 for inserting the center rod 80, and through-holes 38c are formed at the protruding portions, and the pusher members are inserted into the through-holes 38c when the wing portions of adjacent epilating units 30 are arranged crossly.

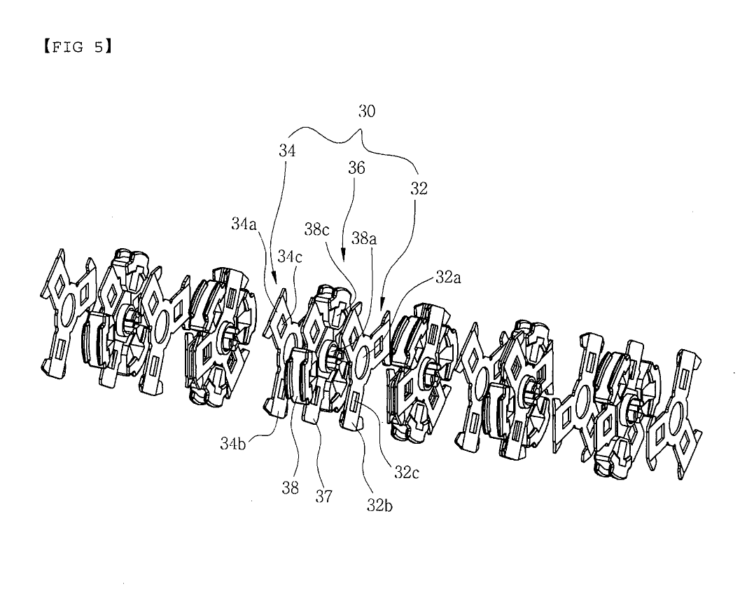

A set of epilating units 30 is comprised of the first pinching plates 32, the second pinching plates 34, and the intermediate members 36. As shown at FIGS. 4 and 5, plural sets of epilating units 30 may be provided. In this case, to prevent the wing portions of adjacent epilating units 30 from being overlapping, it is preferable to arrange the wing portions of adjacent epilating units 30 to be intersected at the angle of 60°, in the rotated state. Accordingly, more hair is pulled out for one revolution of the head A by shortening a cycle for pinching the hair.

The epilating units 30 as described above are pressed and released by the first pusher members 40 and the second pusher members 45, such that the epilating units 30 are driven to pull out the hair.

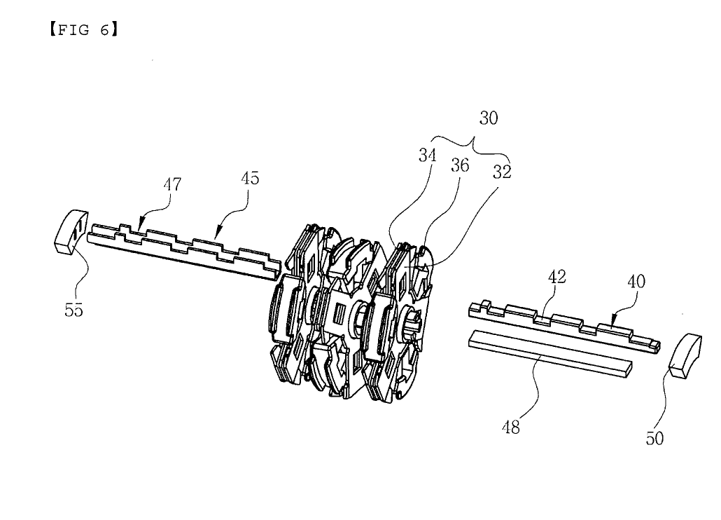

The first pinching plates 32 are pressed inwardly by the first pusher members 40. As shown in FIG. 6, the first pusher members 40 are generally formed by rod member having a rectangle cross-section, and a plurality of recesses 42 are formed at a top surface of the first pusher members 40 at regular interval in a longitudinal direction thereof. Inner-upper sides of the through-holes 32c, 34c and 37c are seated on the recesses 42. Thus, the length of the recess 42 is larger than a sum of thicknesses of the first pinching plates 32, the second pinching plate 34 and the intermediate plate 37 of the epilating unit 30.

The second pusher members 45 are arranged to be overlapped with lower portions of the first push members 40, and fitted in turn into the rotating plates 20 and the through-holes 32c, 34c and 37c of the epilating unit 30, so that the second pinching plates 34 are pressed in opposing direction to the first pinching plates 32. The central portions of the second pusher members 45 are concavely curved in a longitudinal direction thereof such that the first push members 40 are overlapped on the second pusher members from upper side. Therefore, the cross-section shape of the second pusher members 45 is of a concave shape. At both ends of the second pusher members 45, a plurality of recesses 47 are formed at regular interval along a longitudinal direction of the second pusher members 45. Length, depth, size and position of the recesses 47 are the same as those of the recesses 42 of the first push members 40.

A first pressing member 50 and a second pressing member 55 are provided at outsides of the first push members 40 and the second pusher members 45, respectively. Therefore, when rollers 60 are inwardly pressing the first push members 40 and the second pusher members 45, contacting area between the parts can be increased in order to push the push members for a longer time, as described later. The push members and the pressing members 50, 55 can be simply assembled with each other. The pressing members 50, 55 are fixed to ends of the first push members 40 and the second pusher members 45, respectively, which are passed through the rotating plates 20 and protruded outwardly. As a result, outer surfaces of the rotating plates 20 are preferably concaved to cause the pressing members 50 and 55 to be seated at the outer surfaces of the rotating plates 20.

As shown in FIG. 6, it is preferable to further insert the fixing bars 48 into the through-holes 32c, 34c and 37c of the epilating units 30. The fixing bars 48 are means for connecting and fixing adjacent epilating units 30 to prevent them from shaking. The fixing bars 48 are inserted into the through-holes 32c, 34c and 37c of the epilating units 30 along with the first push members 40 and the second pusher members 45, and also are coupled to the through-holes 26 of the rotating plates 20 laterally.

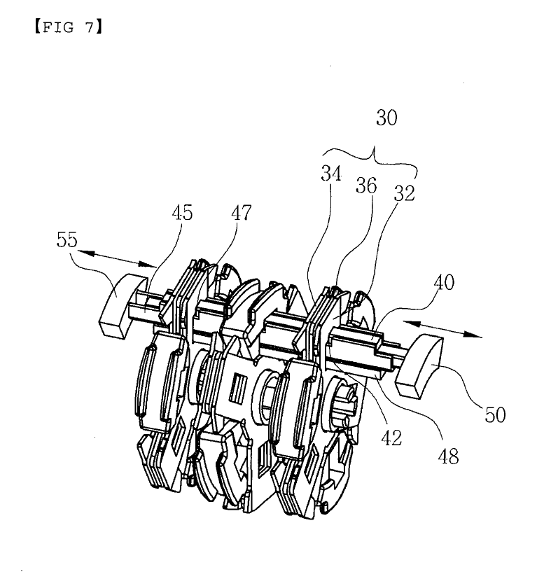

FIG. 7 shows an assembled state of a plurality of epilating units 30, the first push members 40, the second pusher members 45, the first pressing member 50, the second pressing member 55, and the fixing bars 48.

The pair of fixing plates 70 is positioned at outsides of the rotating plates 20, more particularly at outsides of the first pressing member 50 and the second pressing member 55, respectively. Two rollers 60 are installed at inside of the fixing plates 70 at intervals of 180°. The rollers 60 are rotatably installed at roller installation holes 72 formed within the fixing plates 70. The rollers 60 push inwardly on the pressing members 50, 55 to press the push members when the rotating plates 20 and the epilating units 30 are rotated. The fixing plates 70 remain stationary when the rotating plates 20 are rotated. The center rod 80 is fixed to a center portion of the fixing plate 70, and thus the center rod 80 also remains stationary when the rotating plates 20 are rotated.

In order to prevent the fixing plate 70 from rotating, a fixing lever 90 is coupled to outsides of the fixing plates 70. As shown in FIG. 4, the fixing lever 90 has a form of a bar, of which both sides are generally bent at a right angle. Both side portions of the fixing lever 90 are inserted into fixing grooves 74 in the fixing plates 70 to press the fixing plates 70 from outside and are intersected at a center of the fixing plates 70, and a central portion of the fixing lever 90 is inserted and fixed into lever containing grooves 10 of the head fixing case 100. Therefore, the fixing plates 70 can be prevented from rotating, as central portion of the fixing lever 90 is inserted and fixed into the lever containing grooves 10.

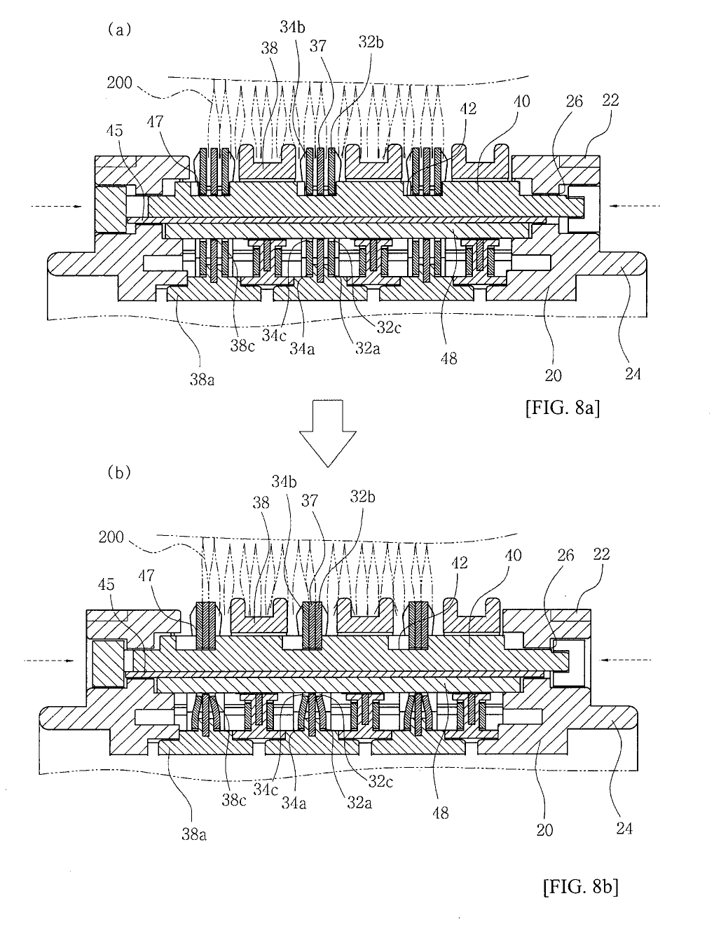

FIG. 8 is cross-sectional view for illustrating operating states of the epilating device including the head according to the present invention, FIG. 8a shows an operating state of the epilating device before pinching of hair, and FIG. 8b shows an operating state of the epilating device after pinching of hair. As shown in FIG. 8a, each of the pinching plates of the epilating units 30 is firstly spaced from each other, so that there is a space between the pinching plates. Thus hair can be inserted into the space. When hair is positioned within the space, the head A rotates, and the rollers 60 push the first push members 40 and the second push members 45. If so, the upper ends of pinching plates having a strength are contacted with both sides of the intermediate pinching plates 37, so that hair can be pinched between the upper ends of pinching plates and both sides of the intermediate pinching plates 37, as shown in FIG. 8b. In the state that hair is pinched by the pinching plates, since the head A rotates continuously, hair can be pulled out by the rotation of the head A. Then, if the head A is further rotated, the pinching plates return to the original positions to create a space therebetween. Hair can be positioned in the created space, and then the above operation can be repeated to carry out the epilating work.

According to the head of the electrically operated type epilating device according to the present invention as described above, the structure of the head can be simple since the pinching plates are comprised of three wing portions. Also, the pinching force for hair can be enhanced by the intermediate member including the intermediate pinching plate between a pair of pinching plates. More hair can be pinched by the pinching plates, since the pinching for hair is made at both sides of the intermediate pinching plate. Further, more hair can be removed for one revolution of the head, as the wing portions of adjacent epilating units are intersected at the angle of 60°, in the rotated state.

The description of the invention set forth herein is illustrative, and is not intended to limit the scope of the invention as set forth in the following claims. For example, while specific speed sensing circuits have been described, the individual components may vary. Other variations and modifications of the embodiments disclosed herein, may be made based on the description set forth herein, without departing from the scope and spirit of the invention as set forth in the following claims.

1. An electrically operated type epilating device, comprising:
a main body housing for containing a motor;
a head fixing case being mounted to an upper portion of the main body housing for fixing a head, and being opened inwardly to expose the head at top thereof;
a motor gear being rotated by the motor;
an intermediate gear being engaged with the motor gear; and
a driving gear being engaged and rotated with the intermediate gear, wherein,
the head A includes:
a pair of rotating plates having teeth engaged and rotated with the driving gear at outer periphery thereof, a hollow shaft at center portion thereof into which a center rod is inserted and passing through it, and through-holes drilled at interval of 60° around a center of the hollow shaft;
a plurality of epilating units being arranged between the rotating plates, wherein each epilating unit having a first pinching plate having a first through-hole at center portion thereof into which the center rod is inserted, three wing portions formed to be protruded outwardly at the first pinching plate at interval of 120° around the through-hole and a second through-hole drilled at a center of the wing portions, a second pinching plate having the same shape with the first pinching plate, and an intermediate member being arranged between the first pinching plate and the second pinching plate;
a first pusher member being in turn fitted into the rotating plates and the epilating units, and pressing inwardly the first pinching plates;
a second pusher member being arranged to be overlapped with lower portions of the first push members, and being fitted in turn into the rotating plates and the epilating unit, pressing the second pinching plates in opposing direction to the first pinching plates;
a roller for inwardly pressing the first push members and the second pusher members;
a pair of fixing plates having roller installation holes for installing the rollers rotatably, and being fixed at ends of the center rod; and
a fixing lever for pressing the fixing plates from outside in order to prevent the fixing plate from rotating, and being inserted and fixed into the head fixing case.

2. The electrically operated type epilating device according to claim 1, wherein the intermediate members being produced by insert injection molding and including an intermediate pinching plates having the same shape and orientation as those of the first pinching plate and the second pinching plate, and a plastic body surrounding a central portion of the intermediate pinching plate from outside and being provided with protruding portions only between wing portions of the intermediate pinching plate to expose the wing portions, a central portion and the protruding portions of the plastic body being formed with through-holes.

3. The electrically operated type epilating device according to claim 1, wherein the first pusher members being formed by rod member of rectangle cross-section, and including a plurality of recesses being formed at a top surface of the first pusher members at regular interval in a longitudinal direction thereof, and wherein the second pusher members having central portions concavely curved in a longitudinal direction thereof such that the first push members are overlapped thereon from upper side, and including a plurality of recesses having the same length, depth, size and position with those of the recesses of the first push members and being formed at regular interval along a longitudinal direction of the second pusher members at both ends of the second pusher members.

4. The electrically operated type epilating device according to claim 2, wherein the first pusher members being formed by rod member of rectangle cross-section, and including a plurality of recesses being formed at a top surface of the first pusher members at regular interval in a longitudinal direction thereof, and wherein the second pusher members having central portions concavely curved in a longitudinal direction thereof such that the first push members are overlapped thereon from upper side, and including a plurality of recesses having the same length, depth, size and position with those of the recesses of the first push members and being formed at regular interval along a longitudinal direction of the second pusher members at both ends of the second pusher members.

5. The electrically operated type epilating device according to claim 3, wherein the head further comprises a first pressing member and a second pressing member at outsides of the first pusher members and the second pusher members for increasing contacting area of the first pusher members and the second pusher members with the rollers when the rollers are inwardly pressing the first push members and the second pusher members, and wherein outer surfaces of the rotating plates being preferably concaved to cause the pressing members to be seated at the outer surfaces of the rotating plates.

6. The electrically operated type epilating device according to claim 3, wherein the head further comprises fixing bars which are inserted into the through-holes to prevent the epilating units from shaking and which cause the rotating plates to rotate along with the epilating units.

7. The electrically operated type epilating device according to claim 5, wherein the epilating units are rotated by 60° and are intersected with adjacent epilating units.
